(19) United States
(12) Patent Application Publication
(10) Pub. No.: US 2017/0050946 A1
(43) Pub. Date: Feb. 23, 2017

(54) ANTIMICROBIALS FROM AN EPIGENETICS BASED FUNGAL METABOLITE SCREENING PROGRAM

(71) Applicants: Danielle H. Demers, Tampa, FL (US); Bill J. Baker, Tampa, FL (US)

(72) Inventors: Danielle H. Demers, Tampa, FL (US); Bill J. Baker, Tampa, FL (US)

(73) Assignee: University of South Florida, Tampa, FL (US)

(21) Appl. No.: 15/220,777

(22) Filed: Jul. 27, 2016

Related U.S. Application Data
(60) Provisional application No. 62/197,233, filed on Jul. 27, 2015.

(51) Int. Cl. C07D 311/58; C12P 17/06

(52) U.S. Cl. CPC C07D 311/58; C12P 17/06; G01N 30/7233

(57) ABSTRACT

Novel antimicrobial compounds and associated methods of development are presented herein.

### CROSS REFERENCE TO RELATED APPLICATIONS

This application is a nonprovisional of and claims priority to U.S. Provisional Patent Application No. 62/197,233, entitled “New Antimicrobials From an Epigenetics Based Fungal Metabolite Screening Program”, filed Jul. 27, 2015, the entire contents of each of which is herein incorporated into this disclosure.

### GOVERNMENTAL SUPPORT

This invention was made with Government support under Grant No. AI103715 awarded by the National Institutes of Health (NIH) and Grant No. AI103673 awarded by the National Institute of Allergy and Infectious Diseases (NIAID). The Government has certain rights in the invention.

### BACKGROUND OF THE INVENTION

Cutaneous Leishmaniasis accounts for one million cases annually with 310 million people being at risk for contraction. Visceral Leishmaniasis accounts for 300,000 cases annually which result in 20,000 deaths annually.

### FIELD OF INVENTION

This invention relates to antimicrobials. Specifically, the invention provides new antimicrobials that are effective against Leishmania donovani.

### SUMMARY OF INVENTION

Fungi are known to produce a wide range of secondary metabolites of interest in drug discovery efforts. In a search for new, bioactive natural products via a fungal metabolite screening program, two new compounds (1, 2) were isolated. Active against an infected macrophage model of the disease causing parasite Leishmania donovani, these compounds are of interest for further drug discovery efforts.

### BRIEF DESCRIPTION OF THE DRAWINGS

For a fuller understanding of the invention, reference should be made to the following detailed description, taken in connection with the accompanying drawings, in which:

FIG. 1 is an image depicting various types of harmful bacteria.

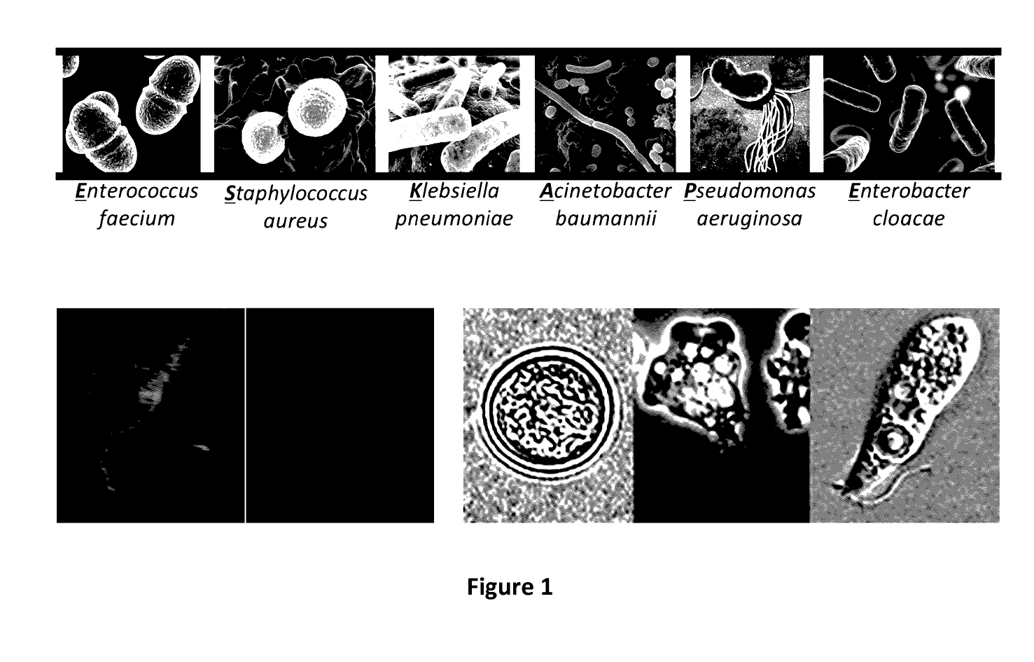

FIG. 2 is an image depicting potential sources of antimicrobials.

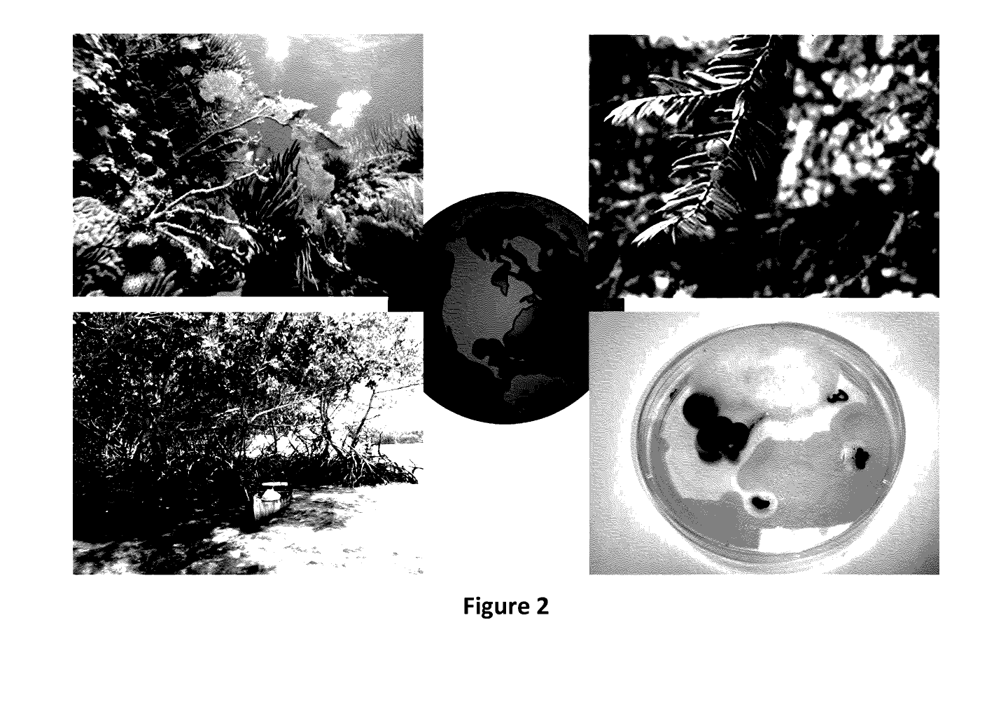

FIG. 3 is an image depicting antibiotic resistance.

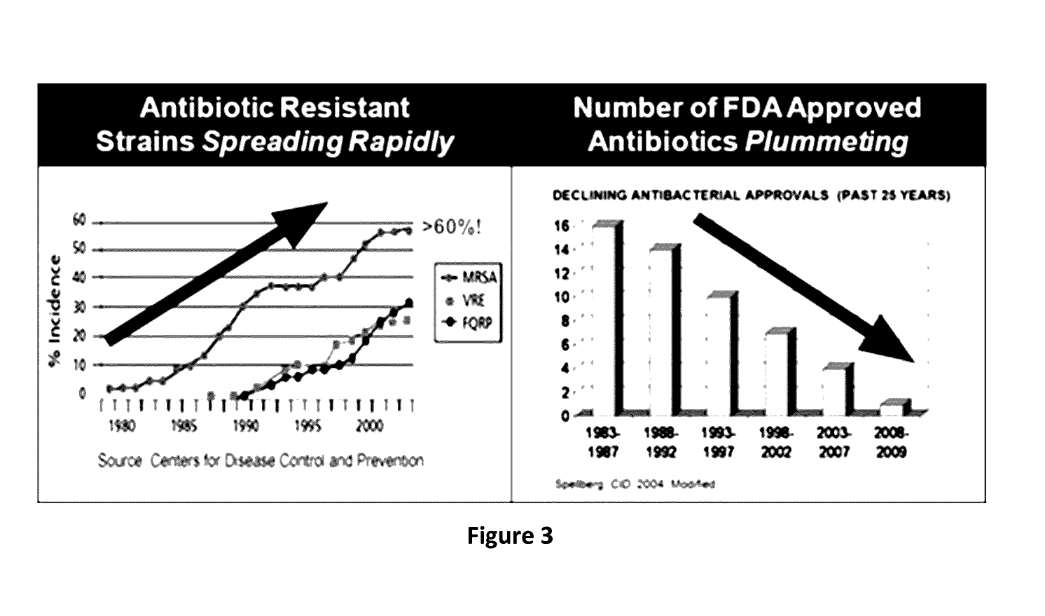

FIG. 4 is an image depicting the endemicity problem.

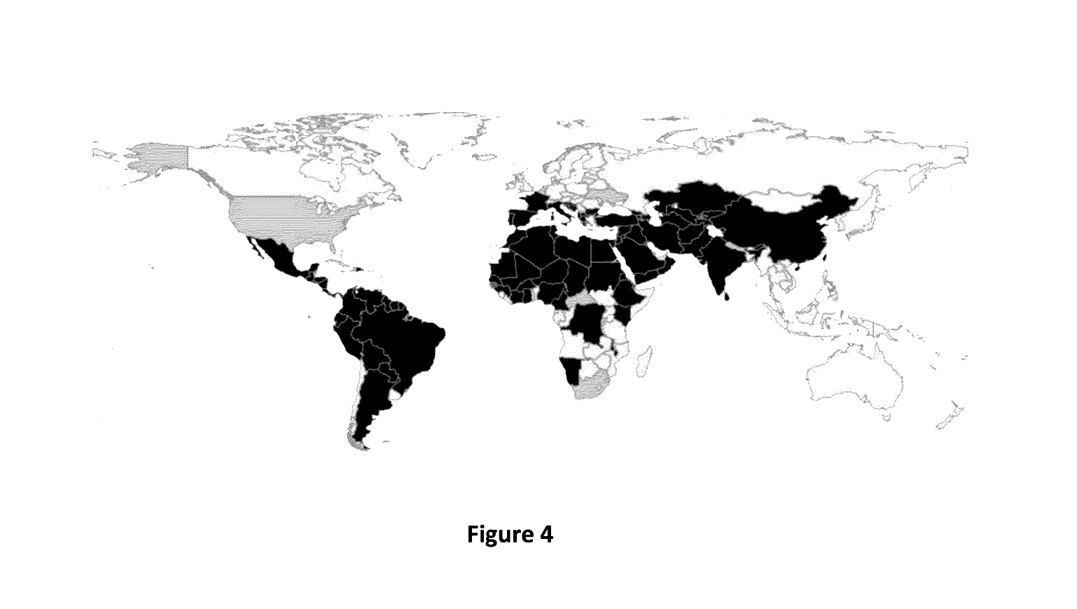

FIG. 5 is an image depicting the no current treatment problem.

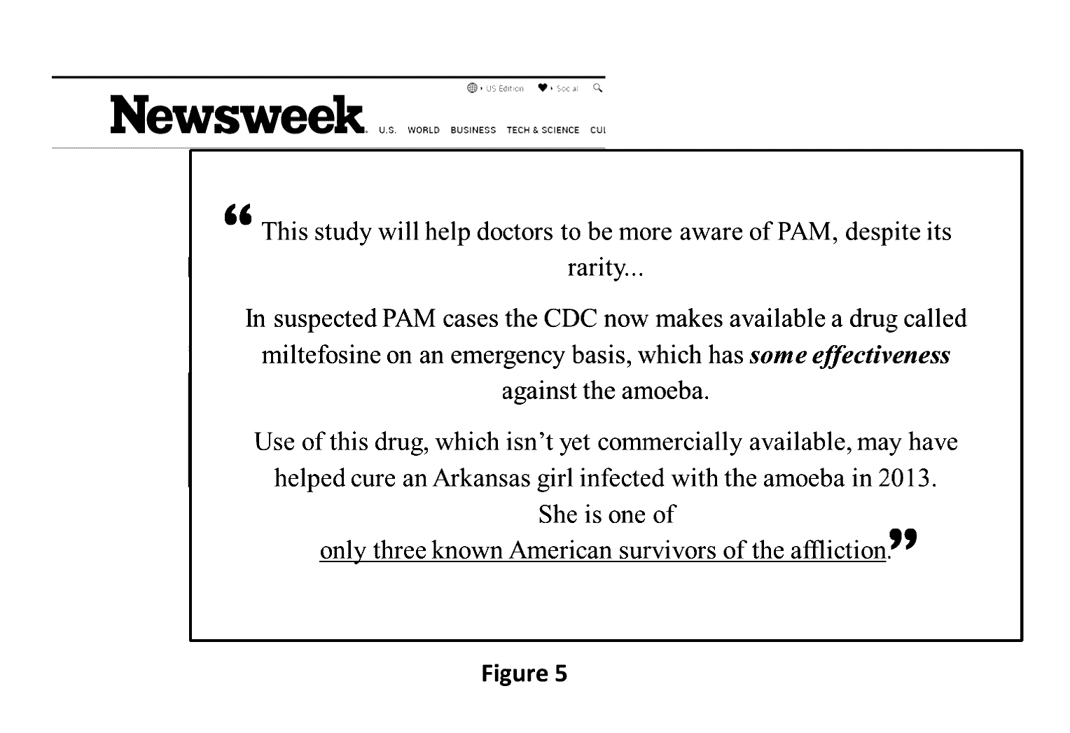

FIG. 6 is an image depicting the drug discovery problem.

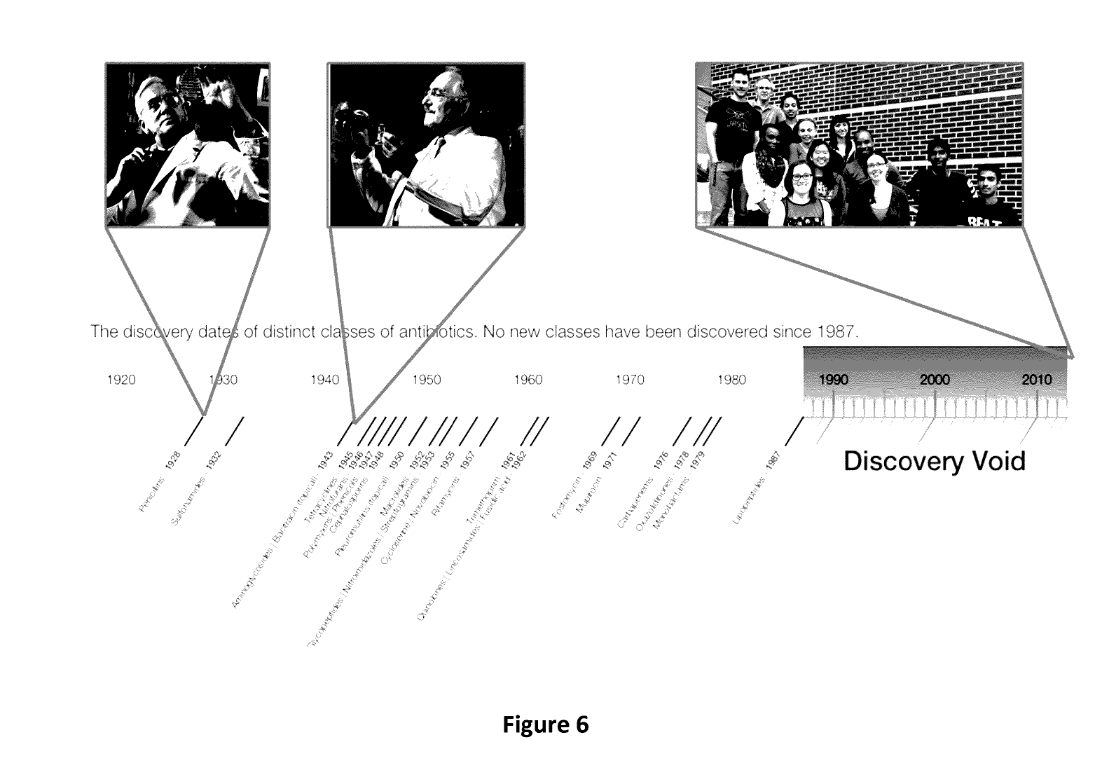

FIG. 7 is an image depicting epigenetic modification.

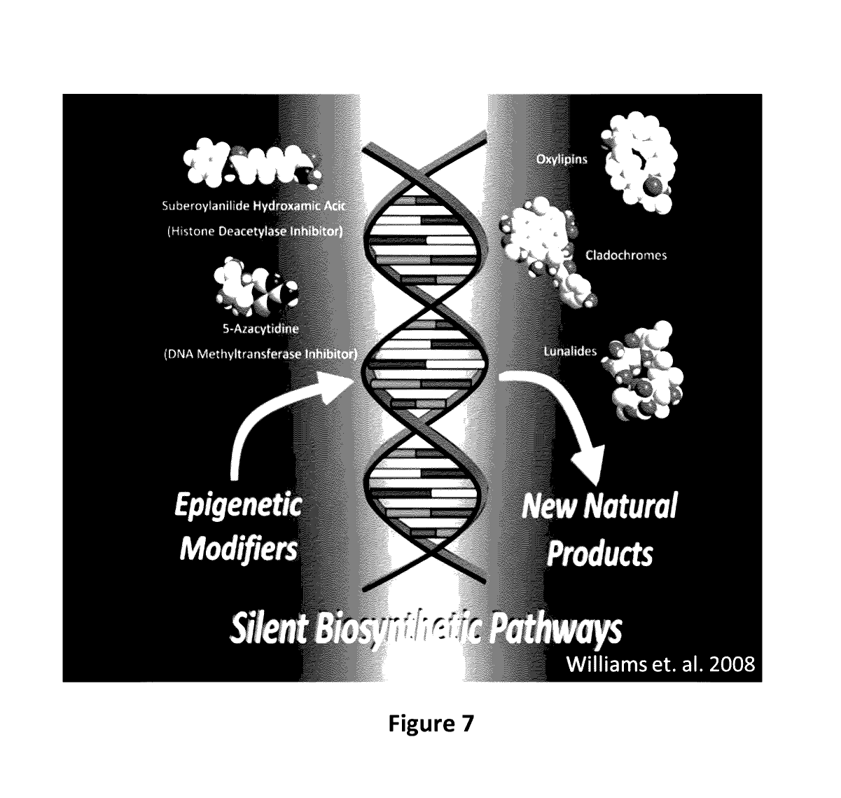

FIG. 8 is an image depicting the screening workflow.

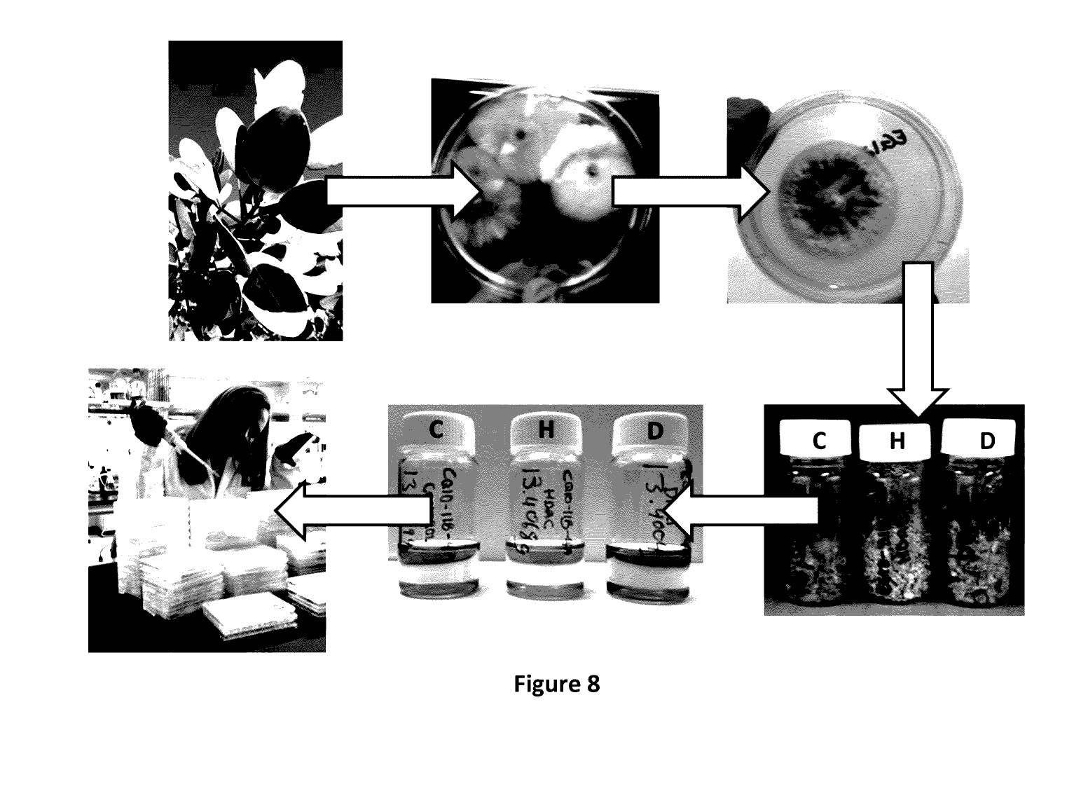

FIG. 9 is an image depicting results from the screening workflow.

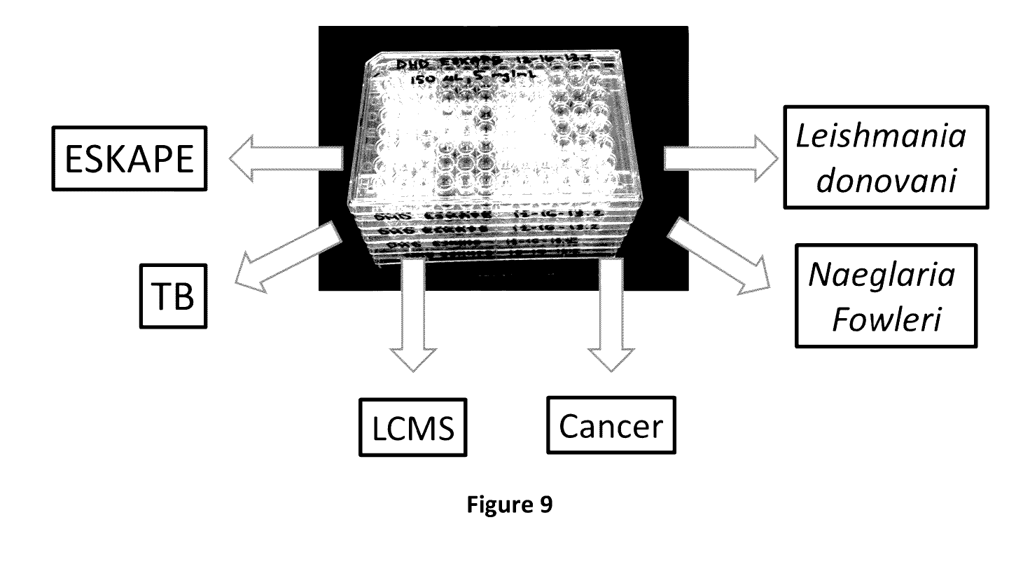

FIG. 10 is an image depicting active organisms.

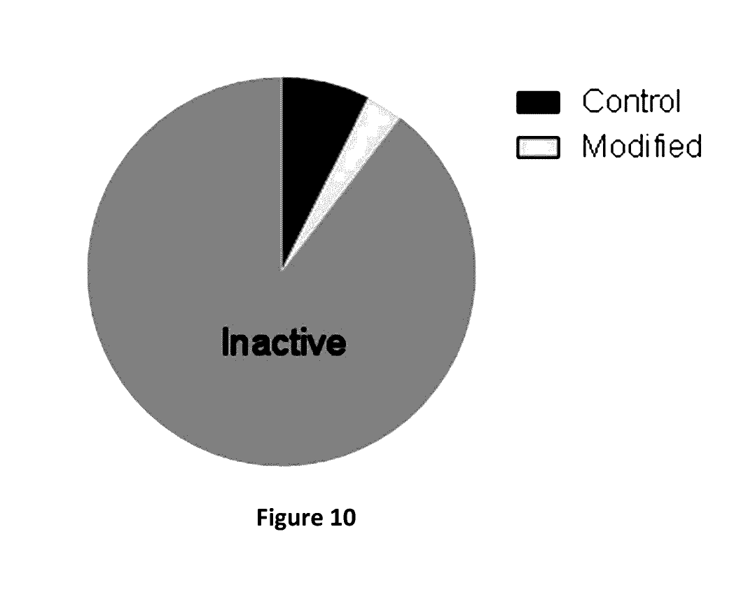

FIG. 11 is an image depicting activity diversity.

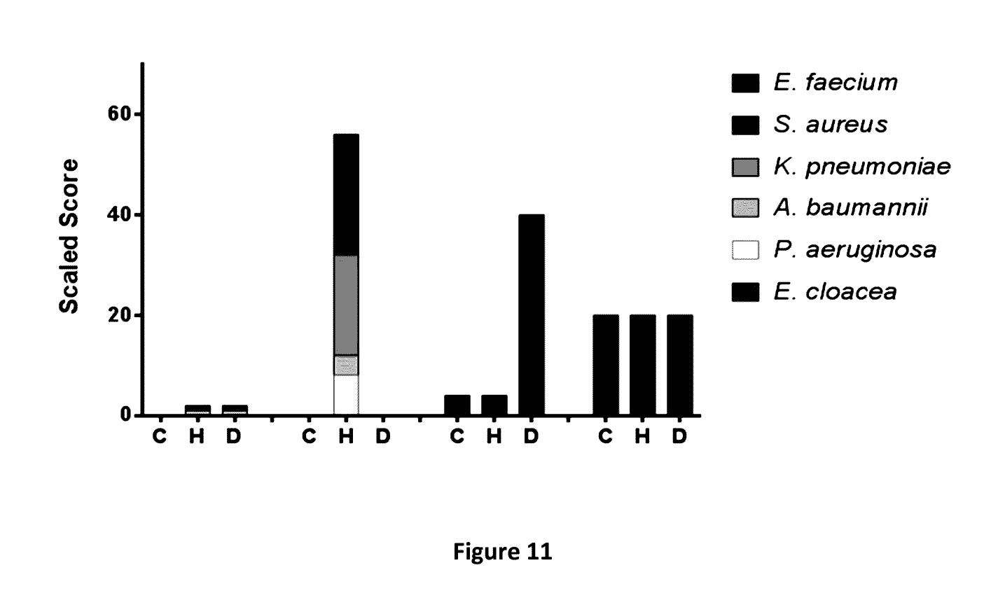

FIG. 12 is an image depicting Bryozoan growing on a stick.

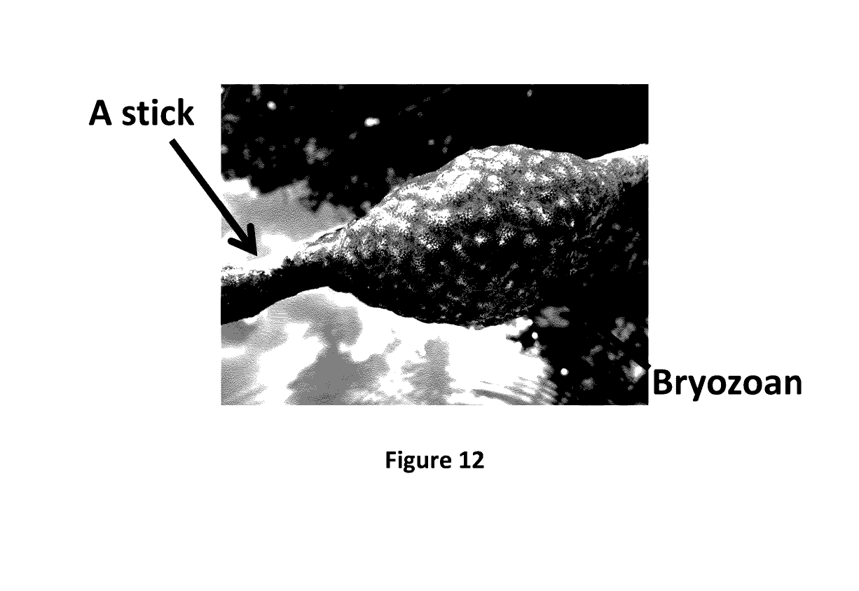

FIG. 13 is an image depicting BB11-2.

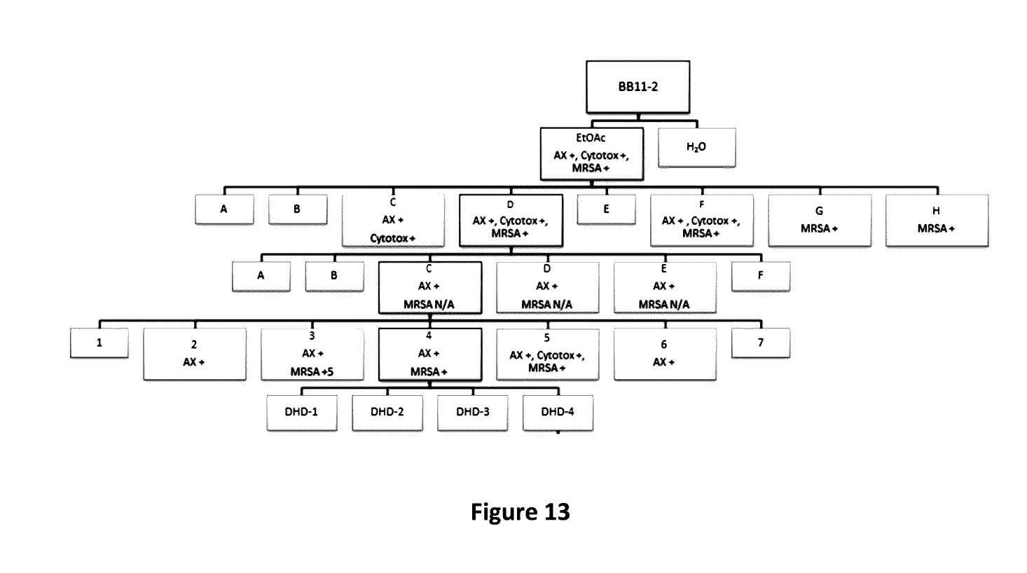

FIG. 14 is an image depicting chemical structures of possible antimicrobials.

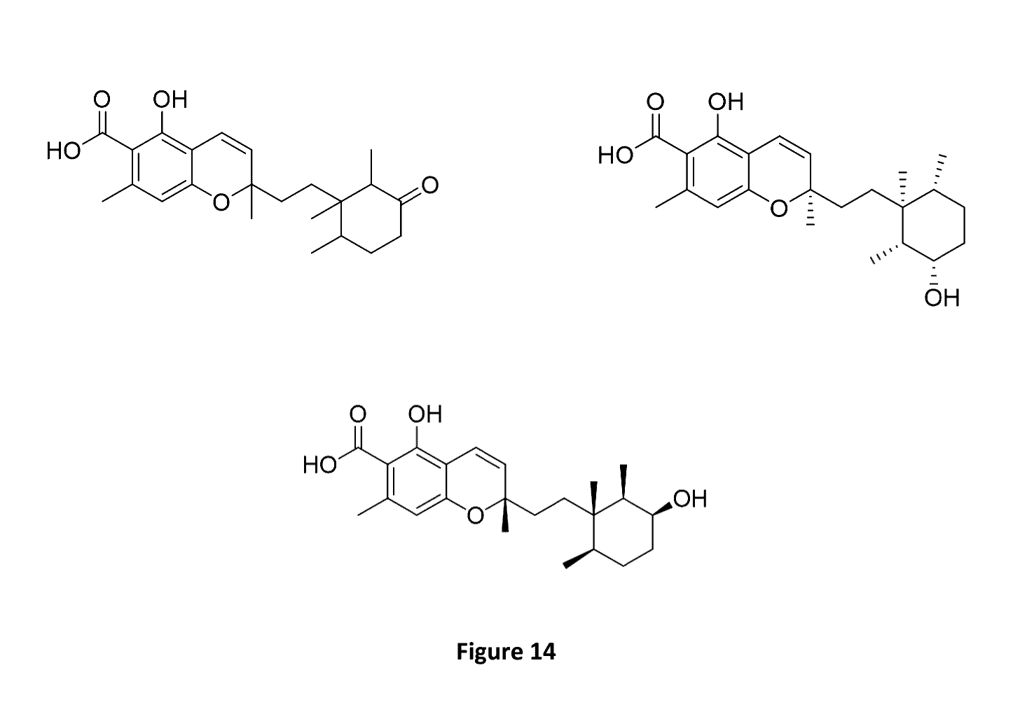

FIG. 15 is an image depicting possible backbone chemical structure of antimicrobials.

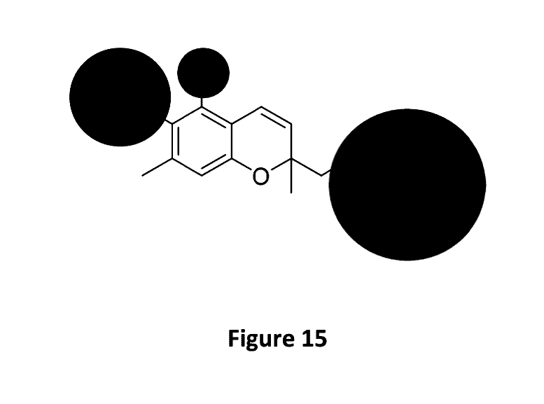

FIG. 16 is an image depicting effectiveness of various proposed compounds against various bacteria.

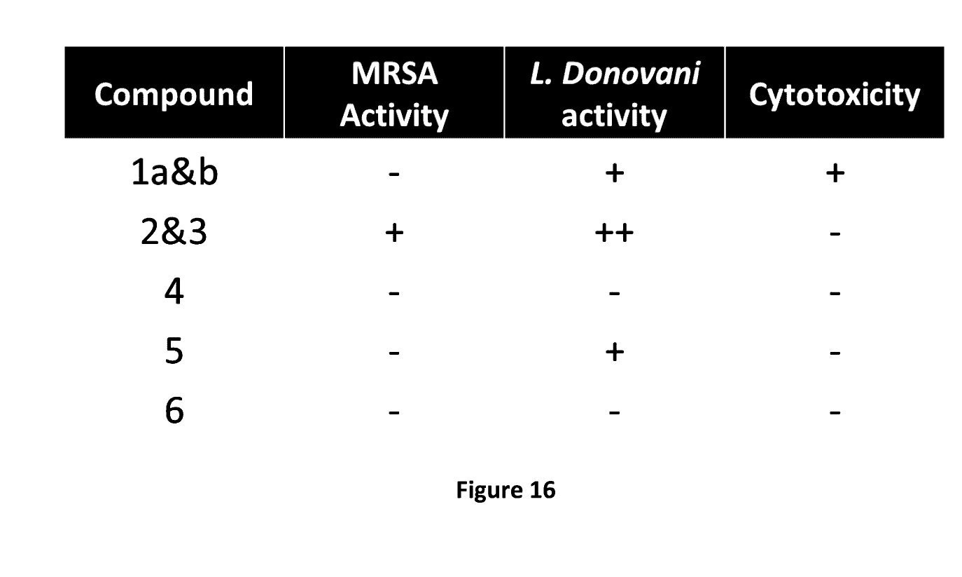

FIG. 17 is an image depicting future work.

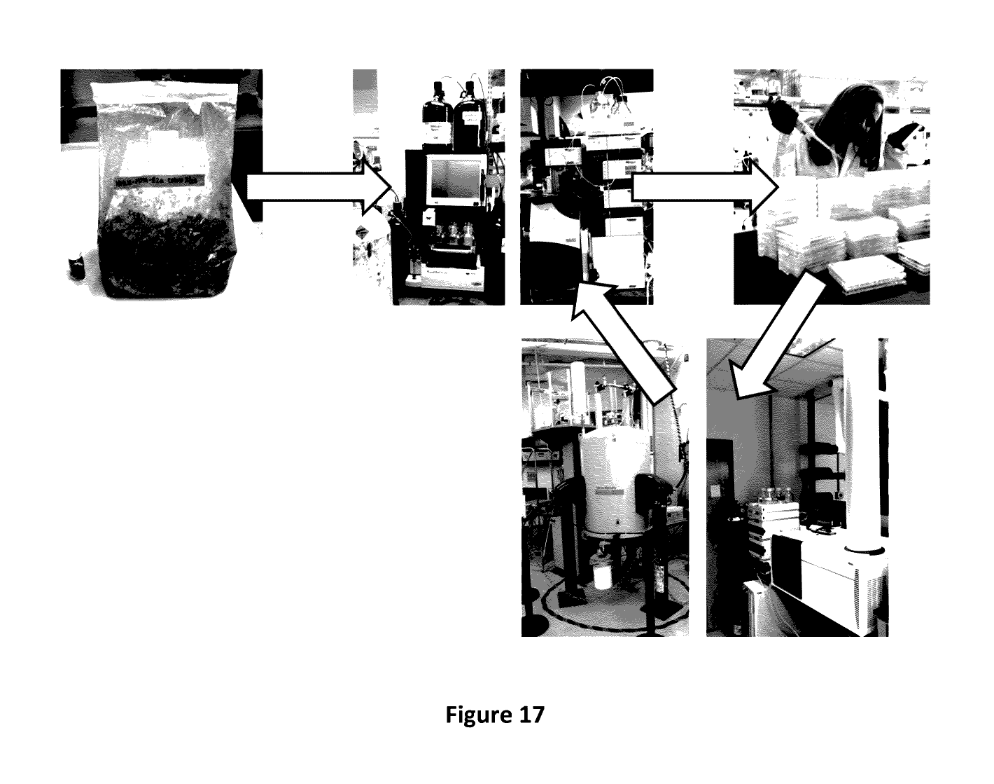

FIG. 18 is an image depicting DHD pure compounds.

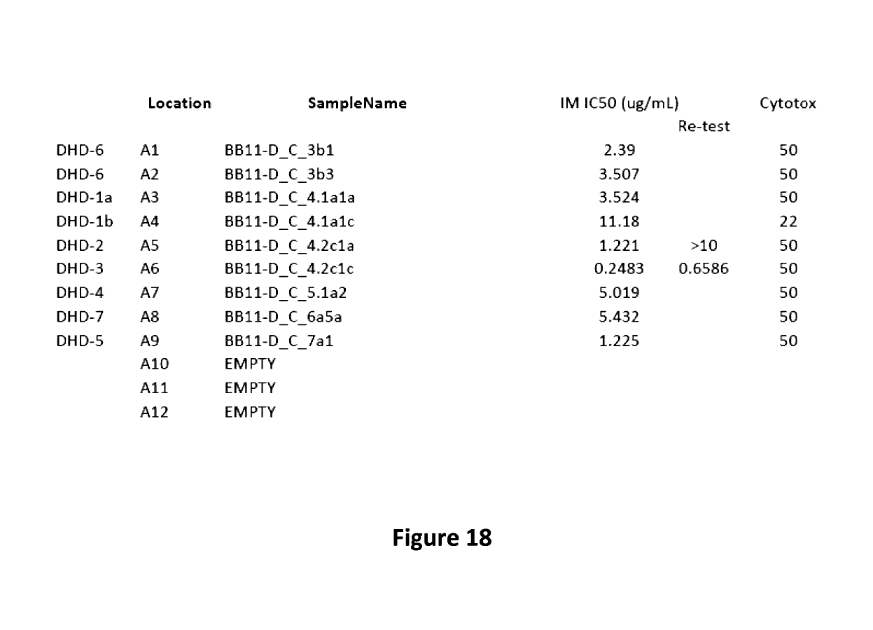

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

### DEFINITIONS

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed in the invention. The upper and lower limits of these smaller ranges may independently be excluded or included within the range. Each range where either, neither, or both limits are included in the smaller ranges are also encompassed by the invention, subject to any specifically excluded limit in the stated range. Where the stated range includes one or both of the limits, ranges excluding either or both of those excluded limits are also included in the invention.

The term “about” as used herein refers to being within an acceptable error range for the particular value as determined by one of ordinary skill in the art, which will depend in part on how the value is measured or determined, i.e. the limitations of the measurement system, i.e. the degree of precision required for a particular purpose. In general, the term “about” refers to being approximately or nearly and in the context of a numerical value or range set forth means ±15% of the numerical value.

As used in the specification and claims, the singular form “a”, “an” and “the” include plural references unless the context clearly dictates otherwise.

Chemistry for terpenoid metabolites can be found in Ellestad, G.A. et al, 1969, herein incorporated by reference into this disclosure in its entirety (Ellestad, G.A., et al., Some new terpenoid metabolites from an unidentified fusarium species, Tetrahedron, 1969, 25(6): 1323-1334). Sesquiterpene hydroquinones and isolation thereof can be found in Shen et al. 2001, herein incorporated by reference by its entirety. (Shen, Y.C., et al., New sesquiterpene hydroquinones from a Taiwanese marine sponge Hippospongia metachromia, 2001, J. Nat. Prod, 64:801-803).

A suite of merosesquiterpenoid natural products were discovered in accordance with the screening procedure outlined in the Disclosure of Screening Format by Danielle Demers and Bill J. Baker. The new compounds, 1 and 2, as well as the known LL-Z1272ε (3) were isolated from an unidentified fungal species obtained from detritus in a retention pond in Tampa, Fla.

1

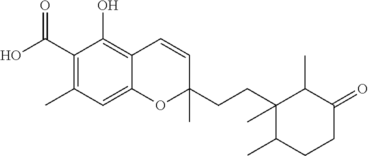 [SMILES: Cc1cc2c(c(O)c1C(=O)O)C=CC(C)(CCC1(C)C(C)CCC(=O)C1C)O2]

2

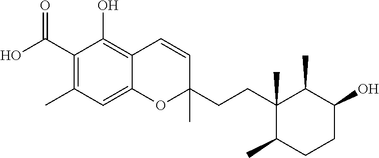 [SMILES: Cc1cc2c(c(O)c1C(=O)O)C=CC(C)(CC[C@@]1(C)[C@H](C)CC[C@H](O)[C@@H]1C)O2]

3

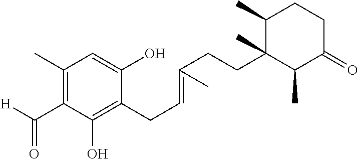 [SMILES: C/C(=C\Cc1c(O)cc(C)c(C=O)c1O)CC[C@@]1(C)[C@H](C)C(=O)CC[C@@H]1C]

| Com- | Molecular | | IM | | Cytotoxicity | S. aureus |
|---|---|---|---|---|---|---|
| pound | Formula | Mass | inhibition | | (J774) | inhibition |
| 1 | C₂₃H₃₀O₅ | 386.2094 | 19 | μM | >0.1 mM | 0.52 mM |
| 2 | C₂₃H₃₂O₅ | 388.2344 | 1.9 | μM | >0.1 mM | 0.52 mM |
| 3 | C₂₃H₃₂O₄ | 372.2417 | 13 | μM | >0.1 mM | none |

Compounds 1-3 were isolated from fungal material grown on rice media for 21 days. Culture media was extracted, and that extract was then partitioned and subjected to liquid chromatography via bioassay and NMR guided isolation. The structures of 1-3 were elucidated using NMR and HRMS. The data obtained for 3 was compared to the available literature and determined to be LL-Z1272ε. 1 and 2 were determined to be new. Bioactivity analysis against Leishmania donovani and murine J774 macrophage cells was executed by the lab of Dr. Dennis Kyle. Analysis of activity against Staphylococcus aureus was completed in the lab of Dr. Les Shaw.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually. Furthermore, where a definition or use of a term in a reference, which is incorporated by reference herein, is inconsistent or contrary to the definition of that term provided herein, the definition of that term provided herein applies and the definition of that term in the reference does not apply.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

While there has been described and illustrated specific embodiments of the invention, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A compound having a formula comprising:

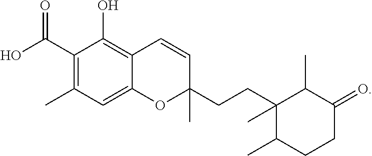 [SMILES: Cc1cc2c(c(O)c1C(=O)O)C=CC(C)(CCC1(C)C(C)CCC(=O)C1C)O2]

2. A compound having a formula comprising:

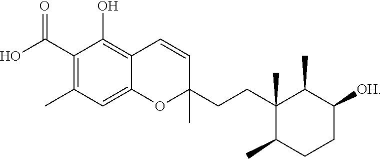 [SMILES: Cc1cc2c(c(O)c1C(=O)O)C=CC(C)(CC[C@@]1(C)[C@H](C)CC[C@H](O)[C@@H]1C)O2]

3. A method of developing an antimicrobial using an epigenetics based fungal metabolite screening methodology comprising:
growing fungal material on media;
isolating the fungal material;
extracting and partitioning culture media;
performing liquid chromatography on the culture media; and
determining structure of the antimicrobial using NMR and HRMS.
